(19) United States
(12) Patent Application Publication
(10) Pub. No.: US 2024/0148644 A1
(43) Pub. Date: May 9, 2024

(54) Gummy Configuration

(71) Applicant: Bradley Zusman, Gresham, OR (US)

(72) Inventor: Bradley Zusman, Gresham, OR (US)

(21) Appl. No.: 18/384,195

(22) Filed: Oct. 26, 2023

Related U.S. Application Data
(60) Provisional application No. 63/423,425, filed on Nov. 7, 2022.

(51) Int. Cl. A61K 9/00

(52) U.S. Cl. CPC A61K 9/0056

(57) ABSTRACT

An edible gummy which may be formed of edible material mixed in a solid solution with medicinal or nutritional supplements. The gummy features a body formed with a plurality of recesses therein extending between a central opening and a sidewall defining a perimeter of the body. The body is easily separated into substantially equal portions having a substantially equal volume of the edible material therein to make dosing of any supplement or medicine contained in the edible material both easy and accurate.

### BACKGROUND OF THE INVENTION

### 1. Field of the Invention

The present invention relates generally to edible soft confectionaries, known conventionally as gummies. More particularly, it relates to the provision of a gummy containing nutritional supplements and/or medications which provides for a substantially equal dosing of the supplement or medication in each of a plurality of separable gummy sections.

### 2. Prior Art

Edible confectionary products have for many years been provided to consumers. Such soft edible products include soft and pliable candy bars as well as soft or pliable edible products commonly known as a gummy or gummies. The term gummy as is employed herein for convenience is meant to include any soft or pliable candy or confectionary which is somewhat elastic and stretches before rupturing into sections or pieces.

Modernly, because such soft confectionary products are easily ingested by both adults and children, the inclusion of nutritional supplements and pharmaceuticals and the like has provided users the ability to ingest supplements and medications by eating such soft confectionaries. Such works well where a single confectionary, be it a soft candy or a gummy, is consistently provided in fixed size so that the dosage can be controlled.

However, such does not work well where there is a single soft confectionary body formed to break apart into sections. This is because it is highly problematic for the user to separate the body of the confectionary having the supplement or pharmaceutical or the like therein into sections with substantially equal doses therein. As is well known, a chocolate bar, having a grid pattern formed therein to depict sections thereof, does not always break apart in sections that are of equal volume so as to provide equal calculated doses of the supplement or medication dissolved within the confectionary material. In the case of semi soft confectionaries, such as chocolate bars, the sections can break irregularly and thereby form such sections in an unequal volume of material forming them.

In the case of gummy type confectionaries, which stretch and easily deform, they are generally only provided in a manner which is not separable. Even where areas to separate from a common gummy body are provided, the elastic nature of gummies and stretching during separation conventionally makes it hard, if not impossible, to form substantially equal sized sections of gummy from a single gummy body. As such, where used to provide doses of nutritional supplements such as vitamins, or to provide a does of a medication such as a cough suppressant, such gummy type confectionaries are conventionally provided in an individual un-separable gummy body of a known size and volume, to insure the appropriate dosage is ingested by the user each time.

The forgoing examples of related art as to separable, soft confectionaries, such as candy bars and gummies and the limitations related therewith, are intended to be illustrative and not exclusive, and they do not imply any limitations on the invention described and claimed herein. Various other limitations in the related art of confectionaries which separate into sections will become apparent to those skilled in the art upon a reading and understanding of the specification below and the accompanying drawings.

### SUMMARY OF THE INVENTION

The device herein disclosed and described enables the formation of soft edible confectionaries, more commonly known as a gummy or gummies, which are employable for a user to ingest nutritional supplements and medications by eating the gummy body. The gummy body is formed of a gummy composition material which solidifies from a liquid to a solid in the form of the mold in which it is placed. When placed in the mold in liquid form, the gummy composite material will cool and harden to form a gummy body in the shape of the mold.

Such gummy confectionaries are widely manufactured and the gummy composite material is formed mostly of gelatin in a mixture with sweeteners such as corn syrup or sucrose along with a starch and water. In addition, minor amounts of coloring and flavoring agents may be added to form the gummy body to have a particular taste or flavor. For example, food acids such as citric acid or malic acid may be added to the gummy composite material so as to provide a tart flavor to the gummy body.

Of course there is a wide variety of contents and recipes for the formation of soft gummy-like confectionaries and all are anticipated as being included herein for use in combination with the supplements and/or medications mixed therewith.

The device and system herein provide for the formation of a gummy body from such gummy composite material in the conventional fashion, in a configuration providing for the formation of sections of the gummy body, which are easily separable therefrom. Each such gummy section in the device herein will have a substantially equal volume of cured gummy composite material as all other sections in the gummy body when engaged thereto, and more importantly, once separated from the other sections.

The provision of such a gummy body which is easily separable into a plurality of sections thereof where each such section has a substantially equal volume of gummy composite material therein, allows for the inclusion of a determined therapeutically effective amount of medication and/or supplement within the gummy composite material. With supplements or medications, so included in the gummy composite material herein, in a volume calculated to yield a determined therapeutically effective amount thereof in each section of the gummy body, when a section is detached from the gummy body, each will have a substantially equal volume of composite material and the therapeutically effective amount of supplement and/or medication therein.

Because the supplements and/or medications which are added to the gummy composite mix are included in a ratio relative to the total volume of the composite material forming the body of the gummy to yield a therapeutically effective amount equally disbursed by mixing into the composite material, the added medications and/or supplements will be evenly disbursed therein. Once the composite material cools and forms to a gummy body shape, the composite material yields a solid solution thereof with the medication and/or supplements equally disbursed throughout the formed gummy body.

In all modes of the gummy body herein, each such gummy body is configured to yield a plurality of sections of the gummy body which are separable as gummy sections. Each such gummy section, either connected to the gummy body or separated therefrom by the user, will have a substantially equal volume of gummy composite with the added supplement and/or medication evenly dissolved therein in the determined therapeutically effective amount. Such a configuration of the gummy body to yield gummy sections where each has a substantially equal amount of gummy composite in a solid solution with the added medication and/or supplement, allows for the accurate delivery of a substantially equal dosage of the medication and/or supplement in the therapeutically effective amount in each individual gummy section.

As such, supplement providers can employ the gummy body herein to provide for the known and proper dosage of one or more supplements to be accurately included in the gummy composite. Because the configuration of the gummy body herein, provides a plurality of gummy body sections, which are easily separated from the gummy body and the rest of the plurality of sections, to form a detached gummy section having a substantially equal volume of gummy composite therein, as all the other gummy sections of the gummy body, each detached gummy section will always have a substantially equal dosage of the dissolved medication and/or supplement therein.

As used herein and in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. Where used herein, if not otherwise respectively defined, the term “substantially” alone or in combination with another term of measurement or volume means plus or minus five percent.

It is the configuration of the formed gummy body of the solid solution of the gummy composite material which provides this equal-sectional utility. Each gummy body is preferably formed of a plurality of gummy sections of substantially the same shape and having substantially an equal volume of gummy composite material therein.

The gummy sections of the gummy body are each defined by a plurality of recesses formed into the first or top surface of the gummy body. It was found in experimentation that, because the gummy composite material, when cured, is soft, compressible, and elastic in nature, that simply tearing the gummy body along the recesses still did not always yield gummy sections of substantially equal volume of the total gummy composite material forming the body.

In multiple configurations of molds, to yield multiple body shapes having substantially equal gummy sections, it was found that running the recesses across the top surface of the gummy body in a manner that they communicate to form breaks or vertical gaps in the circumferential sidewall of the gummy body, did provide for much more accurate removal of the gummy sections from the gummy body. With this communication of the recesses forming vertical gaps within the sidewall, tearing of the sections along the recess yielded separated sections with substantially equal volumes of the gummy composite material forming them. However, the elastic nature of the gummy body formed of the solid solution of gummy composite material, mixed with medications and/or nutritional supplements, still caused gummy sections to separate from the gummy body in a manner where they may not be equal in volume as the remainder of the plurality of gummy sections.

Unexpectedly, the formation of the recesses on the top surface of the gummy body, in a manner where they all communicated with a central opening communicating between the top and bottom of the body, yielded gummy sections of substantially equal volume of the gummy composite material much more predicably. The addition of secondary openings at the opposite end of each recess to the central sidewall of the central opening they communicate with was tried. This was found to provide a best case solution to the stretching and unequal disconnect the gummy sections underwent due to elasticity of the solid solution of gummy composite material.

Consequently, the inclusion of the central opening communicating between the top surface and bottom surface of the gummy body is preferred where substantially equal sections are paramount. The inclusion of secondary openings at the intersection of each recess with the sidewall forming the circumference of the gummy body is especially preferred to yield a more accurate volume in each of the separated gummy sections.

Additionally found to provide for the more equal separation of the elastic and pliable cured gummy composite forming the gummy body, in addition to the secondary openings, is the formation of a slot at the intersection of each recess with a secondary opening. In experimentation, it was found that forming a slot, which communicates through the gummy body at the distal end of each recess with the secondary opening, alleviated the stretching of the gummy body when being torn along a recess from the secondary opening toward the central opening. Instead of initially stretching and then tearing, as occurred without the slot, the gummy body separated along the recess with much less force and much less stretching where the slots were formed. As such, it is especially preferred that slots communicating through the gummy body from the top surface toward or to the bottom surface, opposite thereof, are included.

Finally, during the experimentation with the various configurations, noted herein, for the formation of the recesses separating the gummy sections, it was found that the shape of the bottom surface of the recess itself affected the accurate tearing along the recess to separate a gummy section. A substantially planar surface of the recess worked well. However, it was found that a curved surface or U-shaped surface at the bottom of the recess, where it recesses into the gummy body more in a center area, worked better. Consequently, forming a curved or U-shaped surface at the bottom of each recess would be preferable.

With respect to the above description, before explaining at least one preferred embodiment of the herein disclosed gummy body configured for accurate dosing invention in detail, it is to be understood that the separable gummy providing substantially equal dosing herein is not limited in its application to the details of construction and to the arrangement of the components in the following description or illustrated in the drawings. The invention herein described is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other structures, methods and systems for carrying out the several purposes of the present disclosed gummy nutritional and medical dosing system herein. It is important, therefore, that the claims be regarded as including such equivalent construction and methodology insofar as they do not depart from the spirit and scope of the present invention.

It is an object of this invention to provide a gummy body having a configuration which enables easy separation of the gummy into a plurality of substantially equal gummy sections of the whole body, where each section has a substantially equal volume of gummy composite material forming it as all other sections of the plurality forming the gummy body.

It is a further object of this invention to provide such a separable gummy which is employable for the oral dosing of medication in a manner insuring each section has a substantially equal amount of a supplement or medication, in the determined therapeutically effective amount, as all the other sections in the plurality thereof forming the gummy body.

It is yet another objet of this invention to provide such an easily separable gummy body which is employable for highly accurate oral dosing of nutritional supplements and medications and the like.

Further objects features, and advantages of the present invention, as well as the advantages thereof over existing prior art, which will become apparent from the description to follow, are accomplished by the improvements described in the following detailed description which fully discloses the invention, but should not be considered as placing limitations thereon.

### BRIEF DESCRIPTION OF DRAWING FIGURES

The accompanying drawings, which are incorporated herein and form a part of the specification, illustrate some, but not the only or exclusive examples of embodiments and/or features of the disclosed separable gummy device herein. It is intended that the embodiments and figures herein are to be considered illustrative of the gummy invention having a gummy body with separable sections shown and described system herein, rather than limiting. In the drawings:

FIG. 1 depicts an overhead view of a top surface of a gummy device, as disclosed herein, having a plurality of gummy body sections each of which, when separated from the gummy body, will be substantially equal in size and volume to the other such sections.

FIG. 2 is an overhead perspective view of the gummy device, as in FIG. 1.

FIG. 3 shows an overhead view of the rear surface of the gummy device herein opposite the view of FIG. 1.

FIG. 4 shows a side view at a point of communication of one of a plurality of recesses formed into the gummy device.

FIG. 5 shows another perspective view of the gummy device of FIG. 1 and shows a perspective view of the side shown in FIG. 4.

FIG. 6 depicts an enlarged view of one of the recesses forming the separable portions of the gummy device, as in FIGS. 1 and 5, showing the curved lower surface of the recess communicating into a center opening and a secondary opening formed into the circumference of the gummy body.

FIG. 7 shows a sectional view of the gummy device along line 7-7 of FIG. 5 running through the recess formed therein.

FIG. 8 shows an enlarged view of a side surface of the gummy body showing the recess forming the separation of gummy sections in the gummy body where it communicates with the secondary opening at the circumferential side of the gummy body.

FIG. 9 shows an enlarged view of the rear surface and side of the gummy body of FIG. 1, showing a slot formed into the rear surface communicating with the open area above.

FIG. 10 depicts a rectangular version of the body of the gummy device herein showing the recesses forming four substantially equal sections of the gummy body.

Other aspects of the present gummy body invention shall be more readily understood when considered in conjunction with the accompanying drawings, and the following detailed description, neither of which should be considered limiting.

### DETAILED DESCRIPTION OF THE PREFERRED EMBODIMENTS OF THE INVENTION

In this description, the directional prepositions of up, upwardly, down, downwardly, front, back, top, upper, bottom, lower, left, right and other such terms refer to the device as it is oriented and appears in the drawings and are used for convenience only; they are not intended to be limiting or to imply that the device has to be used or positioned in any particular orientation.

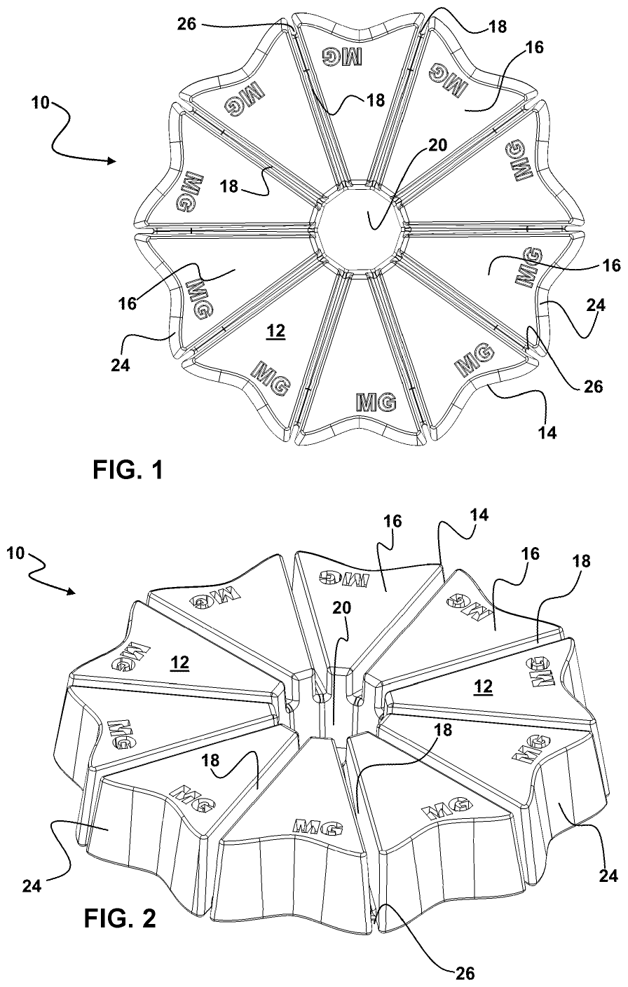

Now referring to drawings in FIGS. 1-10, wherein similar components are identified by like reference numerals, there can be seen in FIGS. 1-2 an overhead view of the gummy device 10 herein. As seen, a top surface 12 of a gummy body 14 forming the gummy device 10 is disclosed. As shown, each gummy body 14 includes a plurality of separable sections 16. As noted herein, each of the sections 16 is preferably formed to be substantially equal in size and volume to all other sections 16 and substantially equal in the volume of gummy composite material.

As noted, during formation of the gummy body 14 from gummy composite material, any medication and/or supplement included into the mixture is mixed into the liquid material before formation of the body 14 so as to be calculated to yield a therapeutically effective equal amount of such in each section 16. Thus, for example, should the therapeutically effective amount be determined to be 10 MG, then in a gummy body 14 with ten sections 16, 100 MG of the supplement and/or medication would be mixed into the gummy composite which will form the gummy body 14 and mixed to a solution. Once cooled and cured the mixture formed to the body 14 will yield a 10 MG amount thereof in each section 16. With the gummy device 10 herein which is formed such that, when torn, each section 16 will have a substantially equal volume of gummy composite therein, the equal dosing in each section 16 is highly predictable.

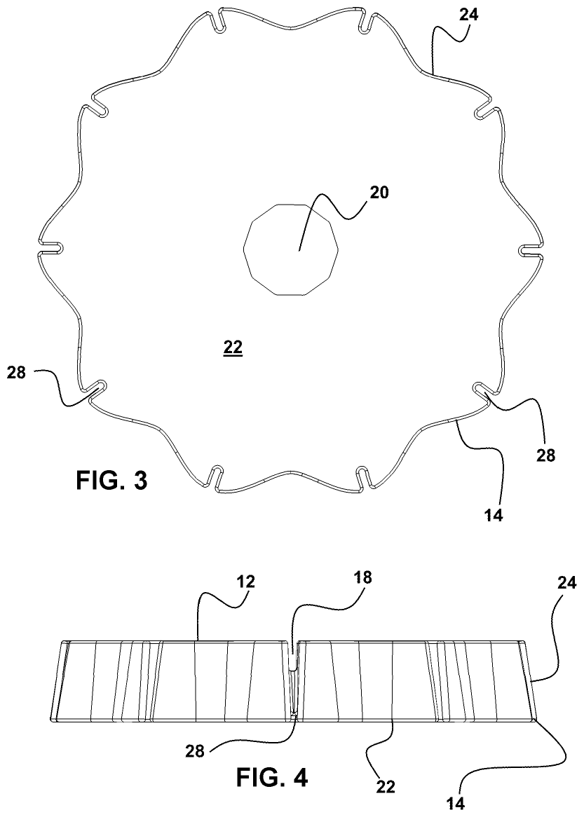

The shape and size of each section 16 is defined by a plurality of recesses 18 formed into the top surface 12 of the gummy body 14. While the body 14 can be formed to divide along the recesses without a central opening 20, such a central opening may be included which communicates between the top surface 12 and an opposite bottom surface 22 (FIG. 3). The inclusion of the central opening 20, as noted, was found to provide a much more even tearing of the sections 16 from the body 14.

Each of the recesses 18 communicate at first ends thereof with the central opening 20 and extend to second ends thereof. Each second end of each recess 18 communicates either with a sidewall 24 of the gummy body 14 or with a secondary opening 26 which communicates into the sidewall 24. As noted, the inclusion of the central opening 20 and the secondary opening 26 was found to yield sections 16 which, when torn from the gummy body 14, were substantially equal in volume as all other sections 16 still attached to the gummy body 14.

FIG. 3 shows an overhead view of the rear or bottom surface 22 of the gummy body 14 of the gummy device 10 herein, which is opposite the view of FIG. 1. As can be seen in FIG. 3, the central opening 20 communicates to and through the bottom surface 22. Also shown are a plurality of slots 28 which communicate through the bottom surface 22 and the top surface from a communication thereof with the endwall 24 of the gummy body 14. These slots 28 will be formed below the secondary opening 26 area where such is included and such is preferred to yield the most accurate equal volume sections 16, when separated.

Shown in FIG. 4 is a side view of the gummy body 14. As shown, one of a plurality of recesses 26 formed into the gummy body 14 is communicating through the sidewall 24 from the first end thereof which, as noted, communicates with the central opening 20 of the gummy body 14 of the gummy device 10.

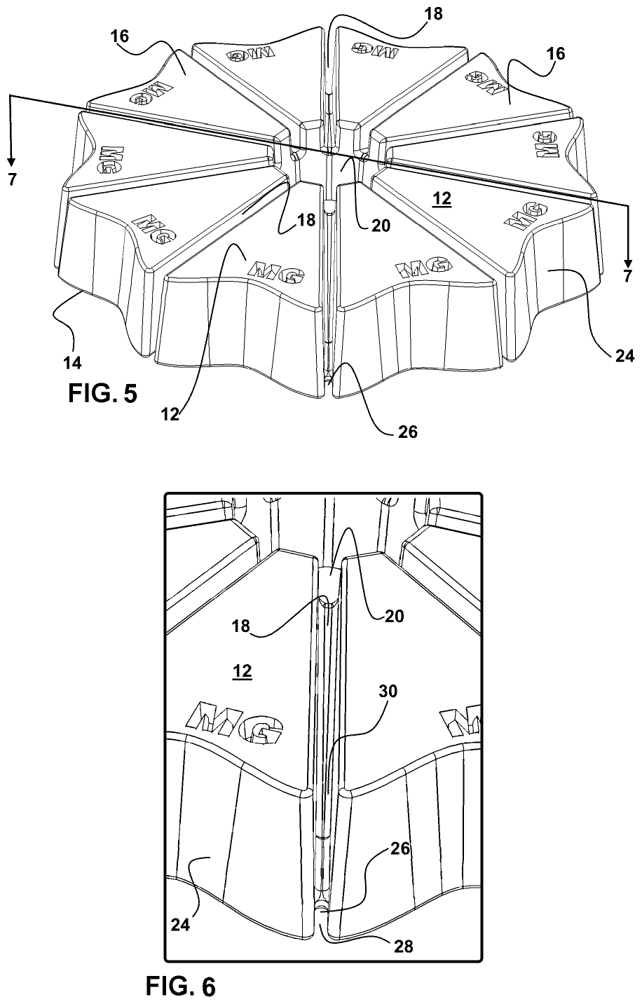

In FIG. 5 is shown another perspective view of the gummy device 10 of FIG. 1 and shows the gummy body 14 in a perspective view. Also shown are the typical communication of each of the recesses 18 with the sidewall 24 along with the slot 28 which communicates through the bottom surface 22 with a secondary opening 20.

Shown in FIG. 6 is an enlarged view of one of the recesses 18 which divide the shape and volume of each section 16. Also shown is the curved lower surface 30 of the recess 18 which is formed into the gummy body 14 from the top surface 12 to the lower surface 30 of the recess 18. Also shown are the secondary opening 26 at the distal end of the recess 18 where the lower surface 30 terminates and the slot 28 communicates through the bottom surface 22 with the secondary opening 26.

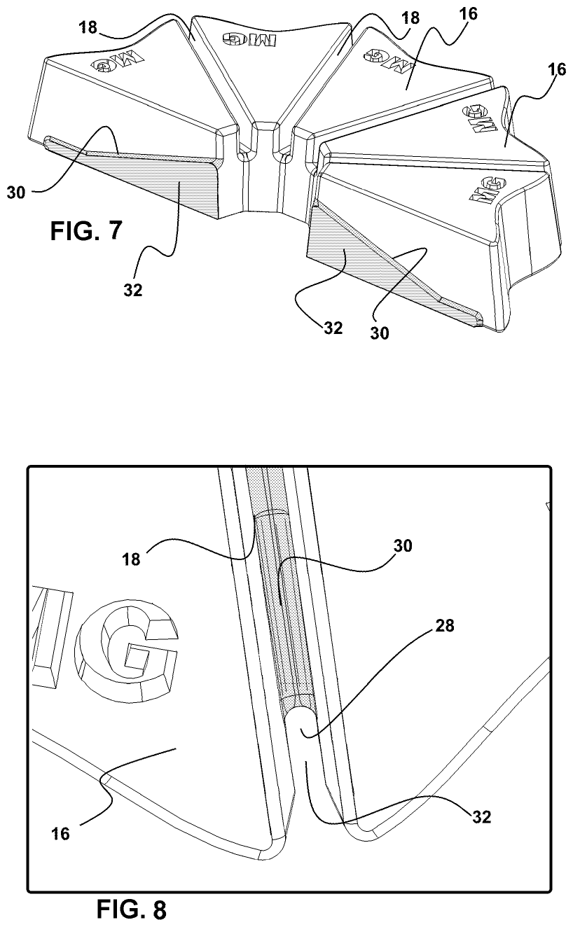

In FIG. 7 is shown the sectional view of the gummy device along line 7-7 of FIG. 5 running through aligned recesses 16 formed into the top surface 12 of the gummy body 14. As can be seen, the lower surface 30 of each of the recesses 16 slants downward from the higher end and thicker body portion 32 at a first end thereof adjacent the central opening 20 to a lower end and thinner body portion 32 adjacent the sidewall 24 of the gummy body 12. This angled lower surface 30 forming separable body areas 30 beneath each lower surface 30 of each recess 18 is preferred as it was found users tearing a section 16 from the gummy body 14, when starting from the sidewall 24 intersection with the recess 16, were able to tear the sections 16 in a fashion to yield much more accurate sections by total volume of each section 16, because it helped avoid uneven tears.

In FIG. 8 is shown an enlarged view of a side of the gummy body 14 where a recess 18, forming the equal separation of gummy sections 16, communicates with the open area formed by the secondary opening 26 which communicates with the sidewall 24 at the circumferential edge of the gummy body 14. Also shown is the lower surface 30, formed at the bottom of each recess 18, which angles downward to communication thereof with the secondary opening 26 and the slot 28 therein. If the secondary opening is included, the end of the lower surface 30 will terminate at the intersection thereof with the slot 28.

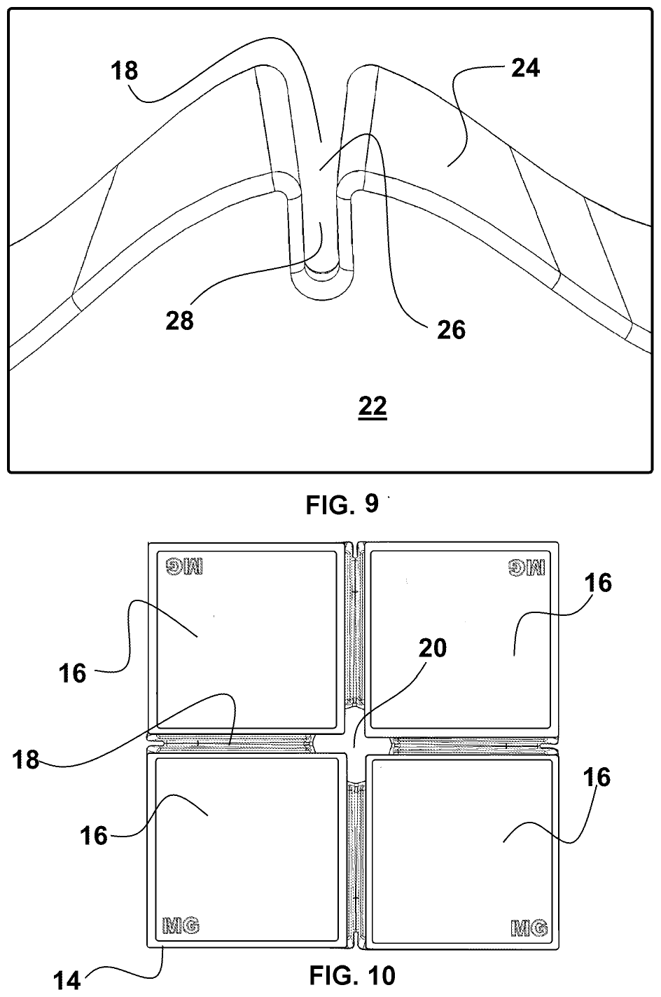

Shown in FIG. 9 is an enlarged view of the rear or bottom surface 22 of the gummy body 14 of FIG. 1. As depicted, the slot 28 is formed through the bottom surface 22 and communicates with the secondary opening 26, if used, or the distal end of the recess 18, if no secondary opening 26 is present. The recess 18 communicates through the sidewall 24 to form an easy point for the user to initiate a tearing of a section 16 from the gummy body 14.

Shown in FIG. 10 is a rectangular version of the gummy body 14 of the gummy device 10 herein. As shown, the recesses 18 extend in the same fashion from the central opening 20 of the gummy body 14. Slots 28 extend through the bottom surface 22 from the opening at the second end of each recess 18. Of course, other shapes of the gummy body 14 are anticipated, so long as each is divisible into sections 16 which, when torn from the body 14, are substantially equal in volume to all others.

While all of the fundamental characteristics and features of the gummy device invention yielding substantially equal tearable sections have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions are included within the scope of the invention as defined by the following claims.

What is claimed is:

1. An edible gummy comprising:
a body formed of edible material, said body having a top surface opposite a bottom surface thereof;
a sidewall of said body communicating in between said top surface and said bottom surface;
a central opening extending between said top surface and said bottom surface;
a plurality of recesses formed into said top surface of said body, said plurality of recesses each having a first end thereof communicating with said central opening and a second end thereof extending to said sidewall;
said plurality of recesses extending into said top surface defining a plurality of body sections therebetween; and
said body separable into substantially equal sections of said edible material by a tearing thereof along said recesses whereby each said section has substantially equal volume of said edible material therein.

2. The edible gummy of claim 1 additionally comprising:
said edible material of said gummy body formed of a solid solution of gummy composite material, mixed with medications and/or nutritional supplements; and
each said section having a substantially equal amount of said medication and/or said nutritional supplements therein.

3. The edible gummy of claim 1 additionally comprising:
said recesses having a lower surface defining a bottom thereof; and
said lower surface being U-shaped.

4. The edible gummy of claim 2 additionally comprising:
said recesses having a lower surface defining a bottom thereof; and
said lower surface being U-shaped.

5. The edible gummy of claim 1 additionally comprising:
a respective said second end of each of said recesses extending into a respective slot formed into said sidewall, said slot extending between said top surface and said bottom surface.

6. The edible gummy of claim 2 additionally comprising:
a respective said second end of each of said recesses extending into a respective slot formed into said sidewall, said slot extending between said top surface and said bottom surface.

7. The edible gummy of claim 3 additionally comprising:
a respective said second end of each of said recesses extending into a respective slot formed into said sidewall, said slot extending between said top surface and said bottom surface.

8. The edible gummy of claim 4 additionally comprising:
a respective said second end of each of said recesses extending into a respective slot formed into said sidewall, said slot extending between said top surface and said bottom surface.

9. The edible gummy of claim 3 additionally comprising:
said lower surface defining each said recess slanting from a higher end of each said recess at said first end thereof to a lower end of each said recess at said second end thereof extending to said slot.

10. The edible gummy of claim 4 additionally comprising:
said lower surface defining each said recess slanting from a higher end of each said recess at said first end thereof to a lower end of each said recess at said second end thereof.

11. The edible gummy of claim 7 additionally comprising:
said lower surface defining each said recess slanting from a higher end of each said recess at said first end thereof to a lower end of each said recess at said second end thereof.

12. The edible gummy of claim 8 additionally comprising:
said lower surface defining each said recess slanting from a higher end of each said recess at said first end thereof to a lower end of each said recess at said second end thereof.

13. The edible gummy of claim 5 additionally comprising:
said plurality of recesses extending radially from said central opening toward said sidewall.

14. The edible gummy of claim 6 additionally comprising:
said plurality of recesses extending radially from said central opening toward said sidewall.
